(19) United States
(12) Patent Application Publication
(10) Pub. No.: US 2026/0354838 A1
(43) Pub. Date: Nov. 12, 2026

(54) Surgical Tool for Different Drilling Speeds

(71) Applicant: Inventitia Laboratories s.r.o., Hradec Králové (CZ)

(72) Inventors: Tomas Hosszu, Skalice (CZ); Ales Bezrouk, Hradec Kralove (CZ)

(73) Assignee: Inventitia Laboratories s.r.o., Hradec Králové (CZ)

(21) Appl. No.: 19/130,301

(22) PCT Filed: Nov. 15, 2023
(86) PCT No.: PCT/CZ2023/050080

§ 371 Date: May 15, 2025

(30) Foreign Application Priority Data
Nov. 15, 2022 (CZ) PV 2022-476

(51) Int. Cl. A61B 17/16; A61B 17/00

(52) U.S. Cl. CPC A61B 17/1615; A61B 17/1628; A61B 2017/0023

(57) ABSTRACT

A surgical tool for different drilling speeds comprises, on one hand, a motor part with a motor and at least one inner shaft of the motor part connected thereto, and, on other hand, at least two replacement parts with a bearing system for connecting a drilling tool and the drilling tool, connected to at least one inner shaft of the replacement part. Furthermore, a gear train is arranged in at least the motor part and/or the replacement parts. The motor part and the replacement parts are adapted for a removable connection, where the inner shaft of the motor part is connected to the inner shaft of the replacement part, wherein this connection transmits radial forces and reduces axial forces.

### TECHNICAL FIELD

The invention relates to the field of disposable surgical drills, particularly to a disposable micro-drill capable of drilling at different speeds, with an integrated battery.

### BACKGROUND OF THE INVENTION

Surgical drills are an important tool for the surgeon's work. In modern medicine, especially in the field of robot-assisted surgery, minimally invasive surgery, microsurgery, etc., high demands are placed on these tools. These are mostly the ability of the tool to work in a wide range of drilling speeds, high precision, high ergonomics with the possibility of both pen and pistol grip, easy handling, and readiness for immediate use, and, especially in the field of surgery on small structures, the lowest possible weight. Another trend in modern medicine is high prevention of the transmission of infectious diseases leading to the use of disposable tools.

In the current state of the art, both corded drills with an external power supply as well as accumulator or battery drills are known. The obvious disadvantage of the corded drills is handling thereof, including other practical disadvantages such as the sterility of the operating space in the case of complex mechanical tools including cabling, as well as in the case of reusable battery or accumulator drills.

The issue of difficult handling cannot be completely eliminated even by the currently available battery drills, which are too heavy with inappropriate weight distribution and non-ergonomic controls especially in the field of surgery on small structures.

Another aspect is the drilling speed and the associated compatibility of the end tools. The type of the drilling tool and the drilling speed must be selected according to the type of the surgical procedure. However, with the disposable drills currently available on the market, speed conversion is unavailable.

For drills in general, there are three basic methods of selecting the speed. The first method is to select the speed using a gearshift mechanism in a gearbox. While this solution theoretically allows the design of the drill with a pen grip and a switch located in the gearbox area to optimise the ergonomics and weight distribution of the drill, it is not yet available on the market because it has several major disadvantages. The main disadvantage is the presence of the gearshift mechanism and all gears at the same time, which significantly increases the weight of the gearbox. The gearshift mechanism and the individual shifting gear segments must also have elements (usually bearings) to ensure the elimination of friction between the gearshift element and the gears and the elimination of the transmission of unwanted axial forces in the gearbox and further into the motor area. Yet, due to the high number of frictional surfaces and the relatively large weight of the inertia masses of the gearbox, the maximum power and operating time of such a system is critically limited in the case of a disposable battery or accumulator micro-drill. However, given the number of individual elements in the shiftable gearbox, even theoretical placement of a switch at the gearbox location is quite complicated and severely limits the ergonomics of the resulting solution.

The second method is to integrate the entire gearbox into a dedicated adapter. This solution ensures full compatibility of the end tool with the rotational speed specific to the particular type of the adapter given by the gears in a particular integrated gearbox, but it is also not yet available on the market because it brings additional disadvantages. This gearbox could also theoretically be made with a switch inserted inside, but this would require integration of a switch separate from the motor part of the drill into each attachment, which would necessarily lead to a reduction in reliability due to a possible effect of contamination of the contacts when moving the adapters to the body of the drill and the risk of sterility violation. In the current known solutions (i.e., without an integrated switch), the adapter is connected to the motor part. This requires placement of elements (usually bearings) eliminating unwanted forces in both parts of the gearbox (input and output) and also in the output part of the motor, which is particularly sensitive to unwanted axial forces and moments of forces. Such a solution therefore has a higher weight and is more burdened by friction, which significantly limits the maximum power and operating time of such a system. Current solutions of such drills with a pen grip also use switches located in areas outside the finger grip and transfer control to the switch via a lever, which can be non-ergonomic and awkward in many cases (e.g., the lever gets caught on the surrounding tools, impaired view during microsurgery or minimally invasive surgery, etc.) and further increases the weight of the drill.

The third solution is to use a gearbox with a plurality of output shafts. Even for these currently used designs, there is no known solution with the switch located ergonomically at the gearbox location. This solution allows full compatibility of the end tool with the rotational speed specific to the particular type of the adapter. The major disadvantage of this solution is the presence of all gears at the gearbox location, which again unnecessarily increases the weight of the system, as only one gear is always in operation. In addition, this solution requires elements (usually bearings) for elimination of unwanted forces on all output axes of the gearbox and also in the adapter being connected. Such a solution significantly increases the frictional forces and inertia mass of the gearbox and, in the case of a battery or accumulator micro-drill, significantly limits the power and operating time of such a system. In a system with a higher number of gears, even the theoretical placement of a switch at the gearbox location is quite complicated and severely limits the ergonomics of the resulting solution.

Based on the above, it is clear that it would be desired to introduce a solution for a disposable surgical micro-drill that allows drilling at different speeds, is easy to use, ergonomic both in terms of grip and control, and is very light (under 200 g). The advantage would be sufficient performance with sufficient operating time to perform the necessary surgical intervention. Such a solution will bring to the surgeon maximum possible comfort and will allow working with maximum precision with minimal negative impact on the patient.

### SUMMARY OF THE INVENTION

Said objective is achieved by a surgical tool for different drilling speeds comprising a motor, a battery, control electronics connected to a switch, a bearing system for connecting a drilling tool, and the drilling tool and at least one gear train. The surgical tool comprises, on the one hand, a motor part with a motor and at least one inner shaft of the motor part connected thereto and, on the other hand, at least two replacement parts with a bearing system for connecting a drilling tool and the drilling tool, connected to at least one inner shaft of the replacement part, furthermore, a gear train is arranged in at least the motor part and/or the replacement parts, wherein the motor part and the replacement parts are adapted for a removable connection, where the inner shaft of the motor part is connected to the inner shaft of the replacement part, wherein this connection transmits radial forces and reduces axial forces.

With regard to the intended applications, it is necessary to design the ergonomics of the tool also for holding the tool as a pen and to keep the total weight of the tool below 200 g. In order to achieve optimum grip ergonomics, the tool design had to be chosen such that the outer dimension of the body of the drill was significantly smaller than 25 mm in the finger area. At the same time, in order to maximize the surgeon's view of the surgical location (especially in the case of minimally invasive surgeries and microsurgeries), it is necessary to narrow the tool body immediately outside the hand behind the grip location as much as possible.

The tool is intended to be disposable with an integrated battery (or accumulator), supplied pre-charged, sterilized, and ready for immediate use out of the box. According to the current requirements for a minimum storage life of 3 years for this type of a medical tool, it is necessary for the tool, even after 3 years of storage without recharging, to be capable of immediate use with sufficient output power for a sufficiently long time needed to perform the surgical intervention.

With the use of modern batteries and motors, it was possible to achieve the desired parameters of the tool by, in the case of the pen grip, placing the (dimensionally larger) battery (or accumulator) in the space above the back of the hand and the (dimensionally smaller) motor with a transmission box in the area of the gripping fingers. This led to an optimal weight distribution and such a position of the center of gravity that the gripped tool does not force the hand to rotate, and at the same time, thanks to a slight extension of the tool body between the part with the motor and the part with the battery (accumulator), a support location was created naturally seated on the hand area between the thumb and the index finger, providing greater grip stability.

In this arrangement, however, the tip of the index finger of the hand that controls the switching of the tool is located in the area of the transmission box. The switching element of the device must be placed in this area. Some systems address this situation by placing the switch body in the area above the back of the hand and controlling the switch with a lever leading from the switch to the location of the tip of the index finger. However, such a lever solution is less ergonomic, can be awkward (e.g., the lever gets caught on the surrounding tools, impaired view during microsurgery or minimally invasive surgery, etc.) and further increases the weight of the drill. Placing the switch outside the transmission box in an undesirable manner increases the diameter of the tool body at the grip location, significantly impairs the grip ergonomics and limits the view of the surgical site. Advancing the entire transmission box outside the grip in front of the tool switch inappropriately changes the center of gravity and weight distribution of the tool and undesirably limits the surgeon's view of the surgical site.

In surgery, different drilling and cutting tools are used for drilling that need to be mounted at different distances from the body of the drill depending on the specific surgical procedure, type of tool, etc. Therefore, it was necessary to choose a design with different end parts of the drill giving the possibility to assemble a drill with a shape adapted to the specific surgical procedure. In order to ensure the accuracy of drilling and to prevent damage to the end parts of the drill, it was necessary to eliminate the transmission of radial and axial load in the distal part of the drill, which is caused by the pressure on the tool during the surgical procedure. At the same time, in order to minimize the weight of the drill and minimize energy losses caused by friction, it was necessary to minimize the number of design parts and their frictional surfaces and to prevent the transmission of unwanted loads inside the gearbox increasing friction. These unwanted loads occur not only due to the pressure of the tool during the surgical procedure but also during the assembly of the individual parts of the drill.

Different types of drilling and cutting tools also require different rotational speeds. The required speeds for individual tool types can vary by more than 30 times.

Therefore, another reason for the development of this product was the need to introduce a disposable tool with the possibility of simple intuitive replacement of the end part of the drill with the desired shape and rotational speed.

The ideal solution seems to be the use of a divided transmission box with integrated control switch, or a dividable tool design. Simply speaking, the inner shaft is divided into two parts, wherein the first part is arranged in the first part of the transmission box in the motor part and the second part in the second part of the transmission box in the replacement part. The first and the second part of the inner shaft are slidingly connected, wherein the sliding connection transmits radial forces, i.e., shaft rotation, but limits the transmission of axial forces. As a result, the driving apparatus of the tool is not excessively loaded by the forces generated by the work of the surgeon and requires use of only one load bearing apparatus in the distal part of the transmission box of the end part of the drill. By using modern batteries (or accumulators) and motors, it is possible to achieve a disposable drill that will be able to use maximum battery power for use during the surgical procedure throughout the storage period by eliminating redundant frictional surfaces and bearings.

The change in speed is preferably achieved by the gear train being arranged in the motor part and having at least two inner shafts of the motor part with different rotational speeds extending therefrom, wherein the inner shaft of each replacement part is adapted in the shaft connection area for selective connection to the designated inner shaft of the motor part.

The inner shaft of the replacement part, i.e., the second part of the transmission box, is specifically terminated at the location of connection to the inner shaft of the motor part in a manner which guarantees connection to the inner shaft which rotates at a speed suitable for the given drilling tool. In practice, this can be a shaped termination of the shaft itself, or use of socket terminals, wherein the terminals are connected to axial clearance and can therefore have limited movement in the longitudinal direction relative to each other. The second part of the transmission box is part of the replacement part of the surgical tool. If the surgeon needs to select a different drilling speed, they remove the first replacement part from the surgical tool and attach the second replacement part. The inner shaft of the second part of the transmission box, in other words the inner shaft of the replacement part, is attached by the shaped termination of a suitable shaft, in other words a shaft with the desired rotational speed. Such a connection remains loose (sliding) in the longitudinal direction of the shafts.

In a preferred embodiment, the shafts of the motor part are in a coaxial, in other words concentric, position, i.e., arranged such that the axes of the shafts overlap, running through the same points in the space. At least one shaft is hollow and rotates about the same axis as the other shaft.

In another embodiment, the shafts can be arranged in parallel, i.e., the shaft axes do not run through the same points but are arranged parallel to each other.

In the case when it is necessary to further adjust the torque of the second part of the inner shaft for work with the drilling attachment, i.e., the transmission of forces from the first part of the driving shaft is adjusted also in the replacement part, the second part of the transmission box comprises a second gear train. The replacement part of the tool acts as a speed regulator, it is the part of the drill that adjusts the speed of the drilling attachment, wherein different drilling attachments can be attached to each replacement part.

The battery and control electronics are preferably arranged in the motor part. For the above reasons, it is possible to place these components in other parts of the body, but in terms of weight distribution and other aspects, placement in the motor part is preferable.

In the case where the body of the drill is adapted for a change of configuration from a pen grip to a pistol grip, the motor part is divided into two parts connected by a locking joint. The division of the motor part is made between the battery and the motor.

Another possibility of converting the drill to a pistol grip drill is that a pistol attachment with contacts and a secondary control switch is fitted on the distal end of the motor part. In this variant, the contacts of the switching circuit are brought out to the distal end of the motor part to a location corresponding to the contacts of the pistol attachment. The contacts of the pistol attachment are connected to the contacts of the drill when the attachment is fitted on the drill and a secondary switch located on the pistol attachment can control the start of the drill motor.

The replacement part of the surgical tool comprises the second part of the transmission box and at the distal end comprises a bearing housing of the system for mounting the drilling tool. The drilling tool can be used for cutting. The drilling/cutting tool may be a fixed part of the replacement part of the surgical tool or may be replaceable. In other variants, the drilling/cutting tool is mounted in the drilling attachment. The output part of the shaft of the replacement part is connected to the handle of the drilling attachment by a bearing system for combined load to reduce both radial and axial load generated by the pressure on the tool during the surgical procedure.

In some embodiments, an additional gear train is arranged in the drilling attachment for further adjustment of the changes of energy transmitted from the source to the drilling tool. Combinations of gear trains included in the first part and/or second part of the transmission box of the drill, or as part of the drilling attachment, provide ample options for designing a drill that meets the requirements for a particular application, which vary in terms of the force exerted on the drill, the required drilling/cutting speed, and the required torque of the shaft.

### DESCRIPTION OF DRAWINGS

A summary of the invention is further clarified by exemplary embodiments thereof, which are described with reference to the accompanying drawings, in which:

FIG. 1 shows schematically a section through the drill and an overall view of the drill of Example 1,

FIG. 2 shows a section and an overall view of the drill of Example 1 with an additional replacement part,

FIG. 3 shows a section through the drill of Example 3,

FIG. 4 shows a detail of the sliding connection between the inner shaft of the motor part and the inner shaft of the replacement part,

FIG. 5 shows a section through the sliding connection between the inner shaft of the motor part and the inner shaft of the replacement part,

FIG. 6 shows a variant of the drill with a pistol attachment.

### EXEMPLARY EMBODIMENTS OF THE INVENTION

The invention will be further clarified by exemplary embodiments with reference to the respective drawings.

### Example 1

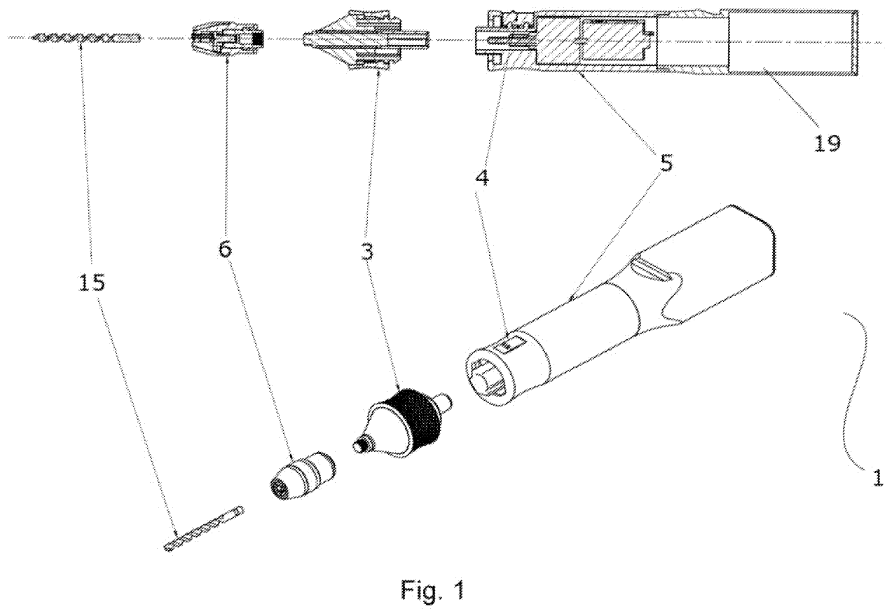

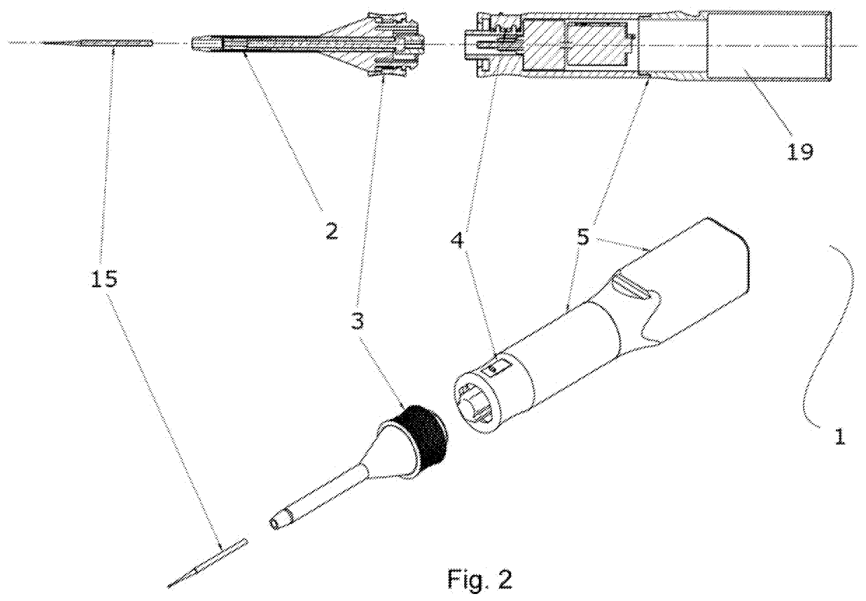

One example of an embodiment of a surgical tool 1 of the present invention is a disposable surgical drill, as shown in FIGS. 1 and 2. The surgical drill comprises a two-piece body with a replaceable front part with a drilling tool 15. The drilling tool 15 may be replaced by a cutting tool 15 depending on the type of surgical procedure required. The cutting and drilling tools perform the same rotational movement, the difference is in the type of the surgical procedure they are used for.

The case of the drill body is plastic, or metal, with sufficient strength to support the drilling tool 15 and hence the entire system of the drill against the pressure generated during the surgical procedure. The motor part 5 of the body, which also comprises the first part 16 of the transmission or gear box, is connected to a replacement part 3 of the body by means of ratchets.

In the motor part 5 of the body, the battery 19 and the motor 14 are arranged in the direction from the outer side. The motor 14 comprises an output shaft Z on which a gear train 10a of the motor part 5 is mounted, the output of which is two internal shafts 13, each with a different rotational speed.

The drill has an elongated body for a pen grip. In a preferred embodiment, by including an additional joint connection, the body can be converted to a pistol grip shape, i.e., a shape approaching the shape of the letter L, if desired. The pistol grip arrangement by means of the joint is preferred particularly for screwdriving of larger screws or for slow drilling where a high torque is required so that the drill can be held in hand and does not rotate, or for the practitioner who requires it for reasons of habit or comfort.

In the motor part 5 of the body, a joint locking connection is arranged at the location between the position of the battery 19 and the motor 14. Alternatively, the pistol grip of the drill can be achieved by using a pistol attachment 20. The pistol attachment 20 is fitted on the distal end of the motor part 5, wherein it comprises contacts for connection to the drill and a secondary switch for controlling the motor 14. The secondary switch is located on the inner part of the pistol attachment 20. This arrangement requires a drill with the contacts for the switch 4 brought out to the surface of the proximal part, that is, in the wall terminating the motor part 5, at locations corresponding to the contacts of the pistol attachment 20. Alternatively, a drill with a pistol attachment that includes an additional battery can be considered.

The battery 19 is located in the peripheral part of the body in the longitudinal direction and preferably the case of the body is designed such as to allow the battery part to be broken off after use, which can thus be separately recycled. The solution is to thin the wall of the case at the locations of the circumferential boundary where the motor part 5 of the body will eventually be broken.

The control switch 4 is connected in electrical circuit to the battery 19 and the motor 14, and is included in the motor part 5 of the body at a location where it will be readily accessible to the user's controlling finger. The position of the switch 4 depends on the weight distribution of the drill. In a preferred embodiment, the switch 4 is located above the location of the sliding connection 12 of the inner shafts 8, 13 in the peripheral part of the motor part 5 towards the replacement part 3.

The inner shafts 13 of the motor part 5 are arranged coaxially, in other words concentrically, thus sharing the course of the longitudinal axis. The first of the inner shafts 13 of the motor part 5 is hollow, and the second inner shaft 13 of the motor part 5 is located therein.

The inner shafts 13 of the motor part 5 are connected to the output shaft Z of the motor 14 preferably by a planetary gearbox. Alternatively, another type of gear train can be used, such as a cycloidal gearbox with high rotational speed reduction, high torsional rigidity and high torque amplification in a single gear.

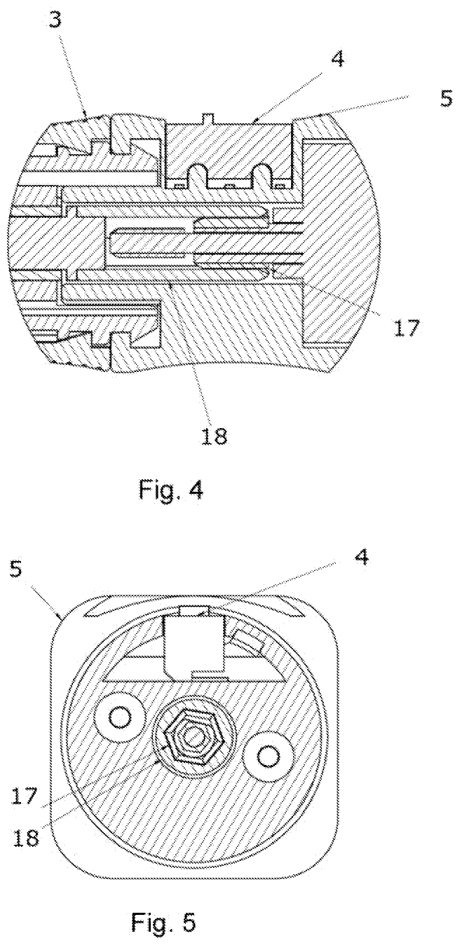

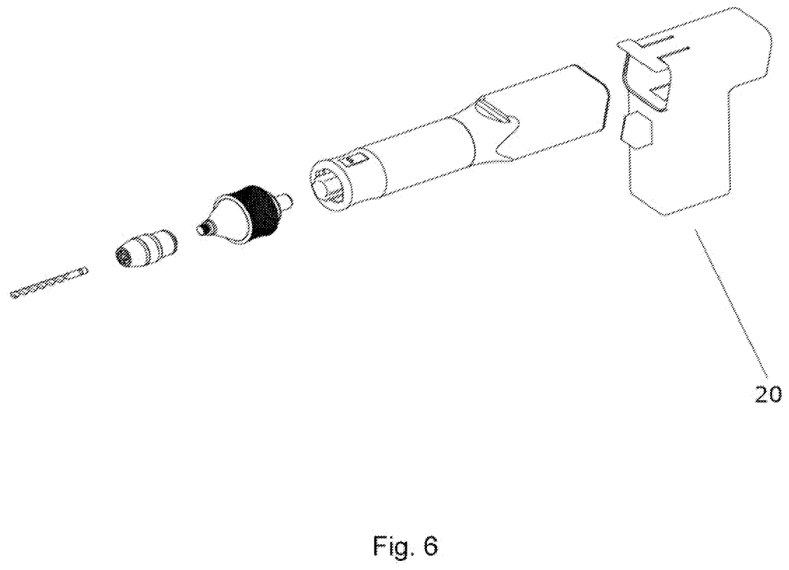

The transmission box is divided perpendicularly to the axis of the shafts. The first part 16 of the transmission box is included in the motor part 5 of the body of the drill, the second part 11 of the transmission box is included in the replacement part 3 of the body of the drill. The transmission box is closed by connecting the motor part 5 and the replacement part 3. The connection of the motor part 5 and the replacement part 3 is removable and immovable, after the connection it does not allow mutual movement of both parts. Therefore, in a preferred embodiment, a ratchet-type connection is chosen. By connecting the motor part 5 and the replacement part 3, the inner shaft 13 of the motor part 5 and the inner shaft 8 of the replacement part 3 are connected at the same time. Detail of the connection 12 of the inner shafts 8, 13 can be seen in FIGS. 4 and 5.

The replacement part 3 of the body of the drill comprises an inner shaft 8 of the replacement part 3, which is mounted on one of the inner shafts 13 of the motor part 5. The inner shafts 8, 13 are profiled into mutually inverted shapes in the area of the sliding connection 12 of the inner shafts 8, 13, i.e., the shaped termination 17 of the inner shaft 13 of the motor part 5 is inverted to the shaped termination 18 of the inner shaft 8 of the replacement part 3 (shown in FIGS. 4 and 5). These ensure automatic engagement of the inner shaft 8 of the replacement part 3 with the terminal of the inner shaft 13 of the motor part 5 which rotates at a speed suitable for the drilling tool 15 of the given replacement part 3 of the body of the drill. The drilling speed of the drilling tool 15 is thus determined by the selected replacement part 3, that is, by the inner shaft 8 of the replacement part 3 which is selectively connected to the particular inner shaft 13 of the motor part 5. The drilling tool 15 thus rotates at a speed dependent on the rotational speed of the given inner shaft 13 of the motor part 5.

The sliding connection is a connection that transmits radial forces and reduces axial forces. In an exemplary arrangement, this is ensured by the shafts being connected by shape but with an axial clearance of at least 0.5 mm. In the case where the shaft ends are designed as an inner and outer hexagon, one shaft is inserted into the other, but the bottom of the inner hexagon is not in contact with the end surface of the outer hexagon, instead there is a free space between them.

Alternatively, the ends of the shafts are not profiled but are provided with terminals, e.g., glued hexagons with different diameters for shafts with different rotational speeds. The first inner shaft is then terminated by an inner hexagon of a specified diameter and the second inner shaft is terminated by an outer hexagon of a specified diameter.

The drilling attachment 6 is housed in the distal end of the replacement part 3 by means of a bearing system 2 for combined load. The drilling tool 15 is fixed in the drilling attachment 6. The drilling attachment 6 may comprise a single fixed tool 15, or the tool 15 may be replaced, or the entire drilling attachment 6 may be replaceable. The most used drilling attachment 6 will be in a form similar to the miniaturized classic universal drill chuck but in an embodiment for medical use or with a lever-type quick-release chuck, and further one for mounting the cutting or drilling tools 15 or for different shapes of drill bit shanks (such as conical, needle, etc.). The drilling attachment 6 may also be plastic with a molded-in drilling tool 15, and the attachment 6 may have a square, slightly conical cavity at the opposite end of the drill bit that is fitted on the square-profile output shaft.

Similarly, the drilling tool 15 itself can be connected to the bearing system 2, without the drilling attachment 6, as shown in FIG. 2. This variant is preferred e.g., in cases of precision milling at high speed, where the whole system is aligned and centered-thus minimizing vibration and frictional losses and making work with the drilling tool 15 more precise.

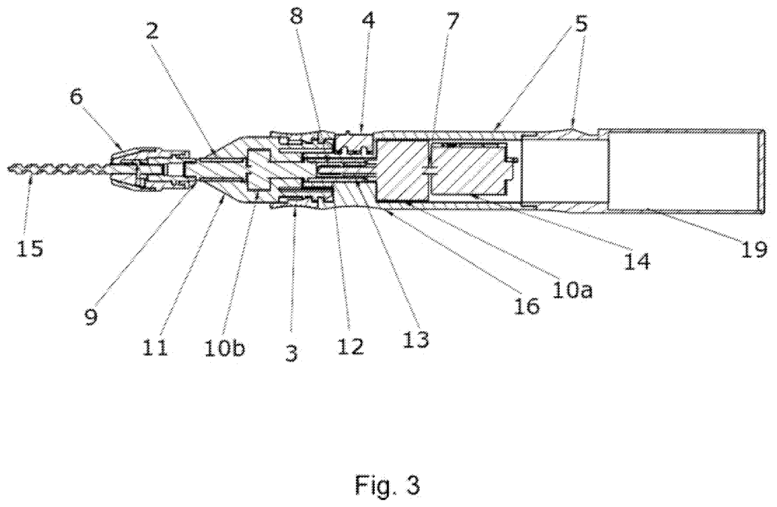

Alternatively, the drilling tool 15 may be connected by a connection member, such as a chuck, to the output shaft 9 of the transmission box, which is connected to the inner shaft 8 of the replacement part 3 by a bearing (can be seen in FIG. 3, where the drilling attachment 6 is connected to the output shaft 9 of the transmission box).

### Example 2

An advantage of the drill with the dividable transmission box is the variability of the design, where the gear train 10 or a part thereof can be placed in the first part 16 of the transmission box and/or in the second part 11 of the transmission box according to the needs of the particular type of the drill. The result is the achievement of the most ergonomic weight distribution of the drill and the optimization of the gears and therefore the drilling speed.

Another exemplary embodiment is a drill with the motor part 5, the replacement part 3, and the drilling tool 15, where the motor part 5 of the drill comprises, in contrast to the drill of Example 1, only one fixed inner shaft 13 connected to the output of the motor 14 by a bearing. The gear train 10b is arranged in the replacement part 3 of the body of the drill. In this embodiment, selective connection of the inner shaft 13 of the motor part 5 and the inner shaft 8 of the replacement part 3 is not necessary, since the motor part 5 comprises only one shaft. The gear train 10b exclusively of the replacement part 3 is then used to change the parameters of the energy transmitted from the motor. However, the change in the resulting drilling speed from the user's point of view is again ensured by the change of the replacement part 3.

Gearboxes of the same type as in the case of the gear train 10a of the motor part 5 can be used as gear train 10b of the replacement part 3, and a gearbox with a bevel gear for concurrent output shafts, or a miniature gearbox with spur toothing for eccentric output shafts, etc., can also be used. As the gear train 10a of the motor part 5, the implementation of a concurrent or eccentric output shaft connection is somewhat disadvantageous.

### Example 3

Depending on the particular type of drill and function and use thereof, it may be preferable to distribute the weight over a greater part of the length of the body of the drill, or this configuration may be required by the position of the individual gears, e.g. if the situation requires a high gear ratio and high rigidity. Then, preferably, a primary gear is used for basic reduction and a secondary gear outside, e.g., with a cycloidal gearbox, for further significant reduction and torsional rigidity, i.e., the torque cannot be easily transmitted from the outer part (in the direction away from the drill bit) into the gearbox 10—e.g. for tightening screws, where the twisting movements are not transmitted back towards the motor part 5 as much when handling the drill.

In this embodiment, one gear train 10a is arranged in the motor part 5 and the other gear train 10b is arranged in the replacement part 3, as shown in FIG. 3. The drill then has the same arrangement as in Example 1, except that the replacement part 3 comprises an additional gear train 10b.

### Example 4

Another embodiment is a drill comprising two inner shafts 13 of the motor part 5, which are arranged in parallel, i.e., their axes are parallel to each other.

The inner shafts 13 of the motor part 5 are connected to the output shaft Z of the motor 5 by the gear train 10a. The output of the motor part 5 is two inner shafts 13 with different rotational speeds. The replacement part 3 comprises an inner shaft 8, the termination of which that is intended to be connected to the inner shaft 13 of the motor part 5 ends at a location corresponding to the position of the termination of the inner shaft 13 of the motor part 5 in the closed state of the transmission box.

The replacement part 3 and the motor part 5 are connected in a way that allows only one position of the connection, i.e., it has for example a guide groove that guides the replacement part 3 to the connection in the corresponding direction, because the inner shaft 8 of the replacement part 3 must engage with the inner shaft 13 of the motor part 5 at the given location, wherein this connection is sliding as well, i.e., it does not transmit axial forces.

### LIST OF REFERENCE SIGNS

1—Surgical tool
2—Bearing system
3—Replacement part
4—Switch
5—Motor part
6—Drilling attachment
7—Output shaft of the motor
8—Inner shaft of the replacement part
9—Output shaft of the transmission box
10—Gear train
10a—Gear train of the motor part
10b—Gear train of the replacement part
11—Second part of the transmission box
12—Connection of inner shafts
13—Inner shaft of the motor part
14—Motor
15—Tool
16—First part of the transmission box
17—Shaped termination of the inner shaft of the motor part
18—Shaped termination of the inner shaft of the replacement part
19—Battery
20—Pistol attachment

1. (canceled)

2. The surgical tool according to claim 10, wherein the gear train is arranged in the motor part and the at least one inner shaft of the motor part comprises at least two inner shafts of the motor part each connected to the gear train and each operable at a different rotational speed, and wherein the inner shaft of each one of the replacement parts is adapted to be selectively connected to a designated one of the inner shafts of the motor part.

3. The surgical tool according to claim 2, wherein the inner shaft of the motor part is configured to be arranged coaxially with the inner shaft of each one of the replacement parts.

4. The surgical tool according to claim 2, wherein the inner shaft of the motor part is configured to be arranged parallel to the inner shaft of each one of the replacement parts.

5. The surgical tool according to claim 10, further comprising a battery and control electronics are arranged in the motor part, and a switch operable to control the control electronics, the battery providing power to drive the motor, the motor being operable by the control electronics via operation of the switch to drive the output shaft, the gear train and the input shaft of the motor part.

6. The surgical tool according to claim 5, wherein the motor part is divided into two parts that are connected by a joint to permit the battery to be separated from the motor.

7. The surgical tool according to claim 5, further comprising a pistol attachment attachable to the motor part, the pistol attachment comprising contacts connectable to the control electronics and a secondary control switch operable to control the control electronics.

8. The surgical tool according to claim 10, further comprising a drilling attachment configured to be connected to the inner shaft of at least one of the replacement parts, the drilling attachment configured to be connected to the drilling tool.

9. The surgical tool according to claim 8, wherein the drilling attachment comprises a further gear train configure the be driven by the at least one inner shaft of each one of the replacement parts to in turn drive the drill tool.

10. A surgical tool comprising:
a motor part comprising:
a motor,
an output shaft connected to the motor,
a gear train of the motor part mounted on the output shaft,
at least one inner shaft of the motor part connected to the gear train and,
at least two replacement parts, each replacement part comprising:
a bearing system for connecting the surgical tool to a drilling tool,
at least one inner shaft. and
at least one gear train configured to connect to the inner shaft or the drilling tool, wherein the motor part is adapted for removable connection to each one of the replacement parts individually, where the inner shaft of the motor part is connectable to the inner shaft of each one of the replacement parts, to transmit rotational forces from the inner shaft of the motor part to the inner shaft of the replacement parts and simultaneously the shafts have movement in a longitudinal direction relative to each other.
